(19) United States
(12) Patent Application Publication
(10) Pub. No.: US 2023/0199315 A1
(43) Pub. Date: Jun. 22, 2023

(54) CAMERA DEVICE

(71) Applicant: OXTI CORPORATION, New Taipei City (TW)

(72) Inventor: Chih-Feng Ho, New Taipei City (TW)

(21) Appl. No.: 17/553,732

(22) Filed: Dec. 16, 2021

(51) Int. Cl. H04N 5/232; H04N 7/18; G03B 17/56; A61B 5/01

(52) U.S. Cl. CPC H04N 5/23299; H04N 7/183; G03B 17/561; A61B 5/01

(57) ABSTRACT

The camera device includes a camera member, a stand and an AI member. The camera member includes a camera and a body-temperature sensing member. The stand supports the camera member, and engages the camera. The AI member is electrically connected to the camera and the body-temperature sensing member, and controls the stand to move the camera according to an image captured by the camera and a temperature value detected by the body-temperature sensing member. The camera device may provide monitoring to a crowd of people within a specific region. The AI member determines the number of people based on the captured image and the detected temperature. When there is a large group of people, the camera is moved backward to cover more people. When there is a small group of people, the camera is moved forward to capture clearer images, thereby achieving effective monitoring.

### BACKGROUND OF THE INVENTION

### (a) Technical Field of the Invention

The present invention is generally related to camera devices, and more particular to a camera device capable of automatic adjustment based on captured images and detected body temperature.

### (b) Description of the Prior Art

As revealed in Taiwan, R.O.C, Patent No. M352057, a video camera includes a camera member and a remote controller. The camera member includes a focus adjustment module, a screen configuration module, a motion detection module, and a partial blocking module. The remote controller transmits various commands to the camera member to adjust, configure, and turn on/off the various function modules of the camera member.

According to this prior teaching, a user can only operate the camera member through the remote controller. The camera member cannot automatically adjust its shooting range and has no additional functionality other than taking images.

### SUMMARY OF THE INVENTION

The present invention discloses a camera device including a camera member, a stand and an AI member. The camera member includes a camera and a body-temperature sensing member. The stand supports the camera member, and engages the camera. The AI member is electrically connected to the camera and the body-temperature sensing member, and controls the stand to move the camera according to an image captured by the camera and a temperature value detected by the body-temperature sensing member.

The stand includes a base. The base has a fastening element or a clamping element.

The AI member includes a control element, a storage element and a motion detection element. The motion detection element may be a gyroscope.

The AI member includes a communication element.

The stand may further include a clamp. The clamp holds the camera.

The camera device may provide monitoring to a crowd of people within a specific region. The AI member determines the number of people present at the scene based on the captured image and the detected temperature. When there is a large group of people, the camera is moved backward to increase the shooting range and to cover more people. When there is a small group of people, the camera is moved forward to narrow the shooting range and to capture clearer images, thereby achieving effective monitoring. The AI member may control the stand to move or to turn, thereby achieving automatic adjustment to the camera's shooting range.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective diagram showing a camera device according to an embodiment of the present invention.

FIG. 2 is a side-view diagram showing support arms of the camera device of FIG. 1 turning relative to each other through a pivot.

FIG. 3 is a side-view diagram showing a clamping element on a base of the camera device of FIG. 1.

FIG. 4 is a perspective diagram showing support arms of the camera device of FIG. 1 spinning through a rotator.

FIG. 5 is a functional block diagram showing the camera device of FIG. 1.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

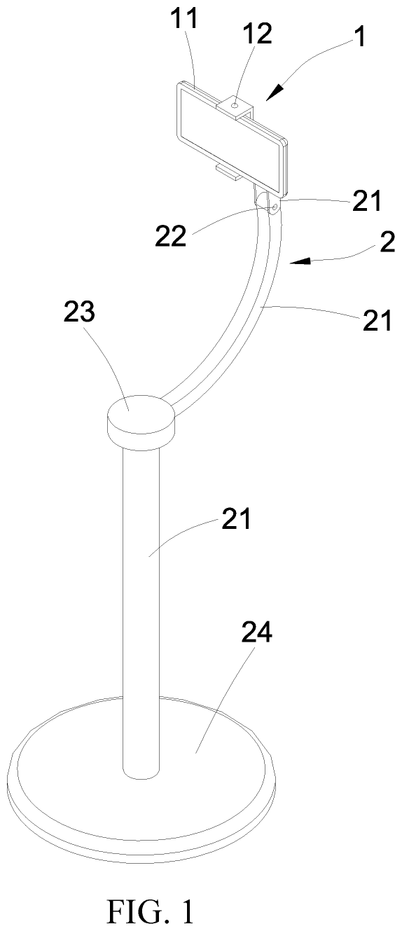

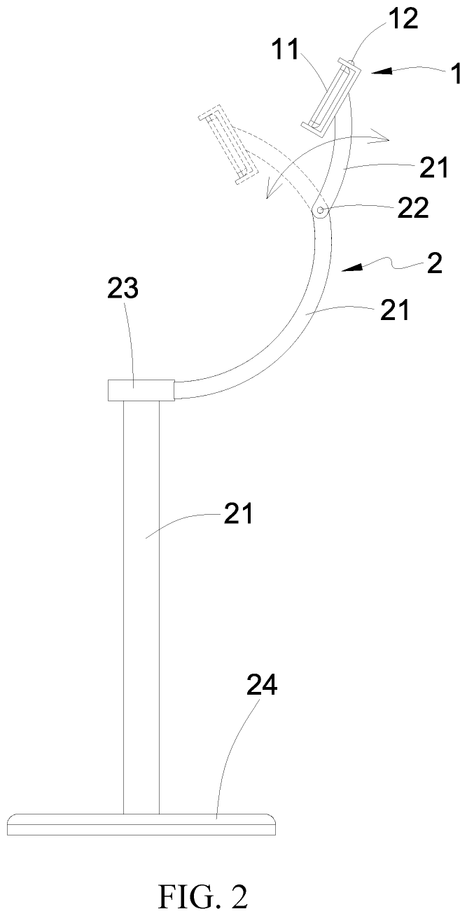

As shown in FIGS. 1 and 2, a camera device according to an embodiment of the present invention includes a camera member 1, a stand 2, and an artificial intelligence (AI) member 3.

As shown in FIG. 1, the camera member 1 includes a camera 11 and a body-temperature sensing member 12. The camera 11 is for capturing images. The camera 11 may be the one on a smart phone, a tablet computer, a notebook computer, a digital cam coder, a digital camera, a webcam, or a screen. The body-temperature sensing member 12 is for detecting a body temperature.

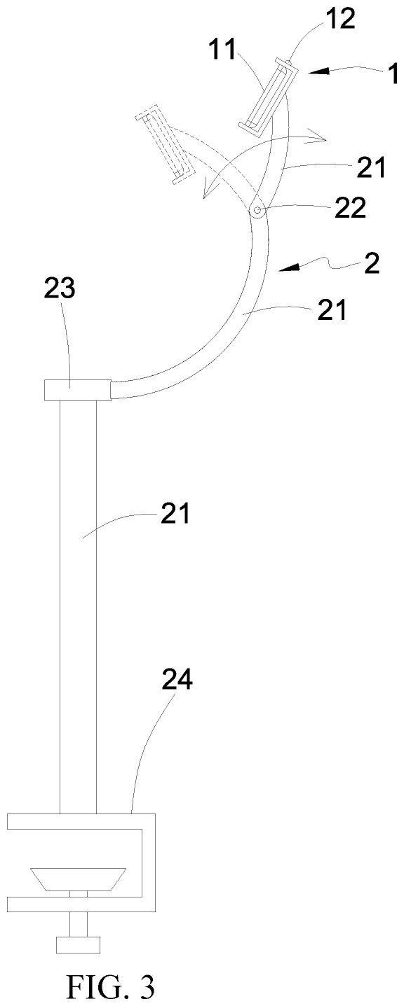

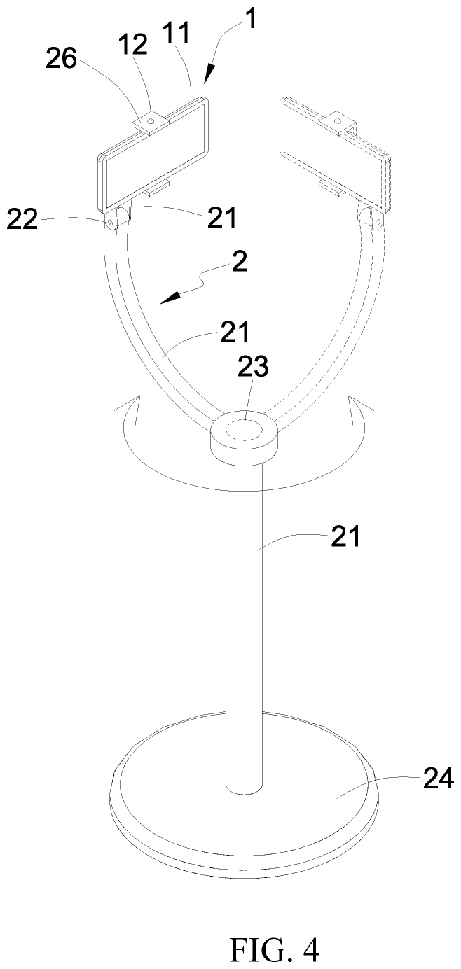

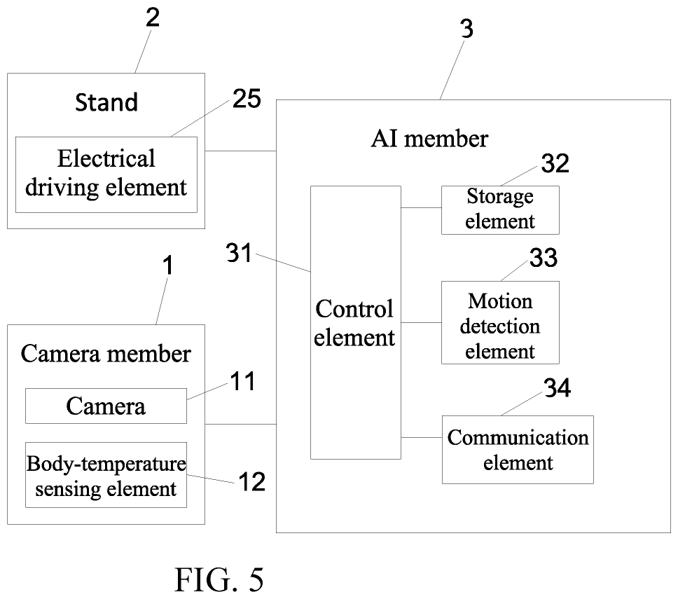

The stand 2 supports the camera member 1, and may move the camera 11. The stand 2 includes a number of support arms 21. The support arms 21 are end-to-end connected by a pivot 22 and a rotator 23. The stand 2 may further include a base 24. The base 24 may include a fastening element (as shown in FIG. 2) or a clamping element (as shown in FIG. 3). The fastening element may be screw, hook-and-loop fastener, or adhesive sticker for fixing the stand 2 onto a plane. Alternatively, the clamping element may be a clamp for clamping the stand 2 to the plane. The plane may be a desktop, a wall plane, or a ground plane. The stand 2 shifts and turns the camera 11 through the pivot 22 and the rotator 23. As shown in FIG. 2, for example, the support arms 21 may turn relative to each other through the pivot 22, tilts the camera member 1 forward or backward, move the camera 11 forward or backward. As shown in FIG. 4, for example, the support arms 21 may turn the stand 2 through the rotator 23, or spin the camera 11. The stand 2 includes an electrical driving element 25 (as shown in FIG. 5). The electrical driving element 25 is for engaging the support arms 21 of the stand 2. As shown in FIG. 4. The stand 2 may also include a clamp 26 connected to a support arm 21. The clamp 26 clamps the camera 11.

As shown in FIG. 1 and FIG. 5, the AI member 3 is electrically connected to the camera 11 and the body-temperature sensing member 12. According to the image captured by the camera 11, and the temperature detected by the body-temperature sensing member 12, the AI member 3 controls the stand 2 to move the camera 11. Specifically, the AI member 3 includes a control element 31, a storage element 32, a motion detection element 33 and a communication element 34. The control element 31 may be a controller, a computer, or a programmable logic controller; the storage element 32 may be a memory or a hard disk; the motion detection element 33 may be a gyroscope; and the communication element 34 may be a wired or wireless transceiver.

The AI member 3 receives at least an image from the camera 11 and at least a temperature value from the body temperature sensing member 12. The control element 31 decodes and analyzes the image to produce data for various human bodies. The storage element 32 has a human body database storing various human body data. The AI member 3, through comparison between the human body data from the image and those stored in the human body database by the control element 31, determines the number of people in the image. In the meantime, the AI member 3 determines whether the temperature value from the body-temperature sensing member 12 through the control element 31 is normal or not. For example, normal human body temperature is within the range 35° C.˜38° C. If the temperature value falls outside the range of 35° C.˜38° C., it is then determined as abnormal. The storage element 32 stores, in addition to the human body database, images from the camera 11 and temperature values from the body temperature sensing member 12. The control element 31 is electrically connected to the electrical driving element 25 of the stand 2 to control the stand 2's operation. In addition, the control element 31 may measure the stand 2's angle and speed through the motion detection element 33 (e.g., a gyroscope) so that the stand 2 may be precisely controlled to move and turn and so that the shooting range of the camera 11 may be automatically adjusted.

The AI member 3 may also be remotely controlled by receiving remote control signals through the communication element 34. The remote control signals may be transmitted through a wired or wireless means to the communication element 34.

Therefore, the camera device may provide monitoring to a crowd of people within a specific region. The AI member 3 determines the number of people based on the captured image and the detected temperature. When there is a large group of people, the control element 31 controls the electrical driving element 25 of the stand 2, where the electrical driving element 25 engages the support arms 21 of the stand 2 to move so that the camera member 1 is tilted backward to increase shooting range and to cover more people. When there is a small group of people, the camera member is tilted forward and the camera is moved forward to reduce shooting range and to capture clearer images, thereby achieving effective monitoring. The camera device may also be applied to epidemic prevention to take images of people of abnormal body temperatures. The images and the detected temperature values are stored in the storage element 32.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the claims of the present invention.

I claim:

1. A camera device, comprising:
a camera member comprising a camera and a body-temperature sensing member;
a stand connecting the camera member and engaging the camera to move; and
an artificial intelligence (AI) member electrically connected to the camera and the body-temperature sensing member, where the AI member controls the stand to move the camera according to an image captured by the camera and a temperature value detected by the body-temperature sensing member.

2. The camera device according to claim 1, wherein the stand comprises a base; and the base comprises a fastening element or a clamping element.

3. The camera device according to claim 1, wherein the AI member comprises a control element, a storage element, and a motion detection element; and the motion detection element is a gyroscope.

4. The camera device according to claim 1, wherein the AI member comprises a communication element.

5. The camera device according to claim 1, wherein the stand further comprises a damp configured to an end of a support arm for holding the camera.
